(19) United States
(12) Patent Application Publication
(10) Pub. No.: US 2021/0256171 A1
(43) Pub. Date: Aug. 19, 2021

(54) PATIENT-SPECIFIC DEVICES AND METHODS FOR ANATOMIC LIGAMENT RECONSTRUCTION OR REPAIR

(71) Applicants: Smith & Nephew, Inc., Memphis, TN (US); Smith & Nephew Orthopaedics AG, Zug (CH); Smith & Nephew Asia Pacific Pte. Limited, Singapore (SG)

(72) Inventor: Ali Hosseini, Quincy, MA (US)

(73) Assignee: Smith & Nephew, Inc., Memphis, TN (US)

(21) Appl. No.: 17/167,637

(22) Filed: Feb. 4, 2021

Related U.S. Application Data
(60) Provisional application No. 62/977,833, filed on Feb. 18, 2020.

(51) Int. Cl. G06F 30/10; A61F 2/08; G16H 50/50; G16H 20/40; G16H 30/40

(52) U.S. Cl. CPC G06F 30/10; A61F 2/0805; G16H 50/50; A61B 5/055; G16H 30/40; A61F 2240/002; G16H 20/40

(57) ABSTRACT

Patient-specific devices are formed from electronic image data taken preoperatively from a patient and used to create a 3-D model of the patient's knee joint. The anatomic location of the anatomic insertion points of the ACL, and hence the anatomic location of a bone tunnel for housing a ligament graft, are then identified on the 3-D model. The anatomic location of the ACL footprint is used to create a 3-D-printed template with apertures corresponding to the footprint of the ACL (or its bundles). The template can be attached to a reusable handle of an existing drill guide for drilling the bone tunnel corresponding to the footprint of the ACL. In other examples, the anatomic location of the ACL footprint is registered and mapped onto a real-time computer display of the patient's bone during the ligament reconstruction.

### CROSS-REFERENCE TO RELATED APPLICATIONS

This application claims priority to and benefit of U.S. Provisional Application No. 62/977,833, filed Feb. 18, 2020, entitled PATIENT-SPECIFIC DEVICES AND METHODS FOR ANATOMIC LIGAMENT RECONSTRUCTION OR REPAIR, the entire contents of which are incorporated herein by reference for all purposes.

### FIELD

The present disclosure relates to devices and methods for surgical reconstruction or repair of joint ligaments using patient-specific data.

### BACKGROUND

A ligament, such as an anterior cruciate ligament (ACL), that has ruptured and is non-repairable, is generally replaced arthroscopically by a tissue graft. The replacement graft is usually implanted by securing one end of the graft in a bone tunnel formed within the femur, and securing the other end of the graft in a bone tunnel formed in the tibia. In many cases, the function of the reconstructed knee joint is dependent on the anatomic location of the tunnel drilled through the femur and/or the tibia to house the tissue graft. For example, grafts placed too far anteriorly on the femur are reportedly a common cause of failure in ACL reconstruction. If the tunnel location is placed on anatomic footprint of the native ACL, the physiological outcome of the operation is greatly improved and reduces the need for a potential revision ACL reconstruction. However, accurate determination of the ACL footprint during arthroscopic ligament reconstruction can be challenging, especially for more junior surgeons. The location of the ACL footprint may vary among patients based on gender, height and other features, while many current devices used to predict the ACL footprint are based on an average footprint size and location and used for all patients. These devices could create up to a few millimeters of error in predicting the native ACL footprint. Therefore, it is desirable to have devices and methods for more accurate placement of the femoral and/or tibial tunnels to reduce the incidence of graft failure and/or long-term degeneration after ligament reconstruction.

### SUMMARY

Described herein patient-specific devices and methods designed to eliminate misplacement of an ACL graft tunnel relative to the native ACL insertion points on the corresponding bone. Initially, electronic image data is taken preoperatively from the patient and used to create a 3-D model of the patient's knee joint. The location of the anatomic insertion points of the ACL, and hence the location of the bone tunnel for housing a ligament graft, are then identified on the 3-D model. In some examples, the location of the ACL footprint is used to create a 3-D printed template with apertures corresponding to the footprint of the ACL (or its bundles). The template can be attached to a reusable handle of an existing drill guide for drilling the bone tunnel corresponding to the footprint of the ACL. In other examples, the location of the ACL footprint is registered and mapped onto a real-time computer display of the patient's bone during the ligament reconstruction. The surgeon can use the display as a reference to decide the final location of the ACL footprint before placing the graft tunnel. Advantageously, both methods provide the surgeon with patient-specific data for accurately determining the location of a bone tunnel for housing a ligament graft on the femoral and/or tibial bones.

Further examples of the methods and devices of this disclosure may include one or more of the following, in any suitable combination.

In examples, a method of making a surgical instrument of this disclosure includes obtaining electronic image data of a joint, including at least one bone, of a patient. Using the electronic image data, a 3-D model of the at least one bone is created. Using the 3-D model, at least one anatomic insertion point of a ligament on the at least one bone is determined. Based on the at least one anatomic insertion point, an anatomic location of a tunnel through the at least one bone is determined for housing a graft. Based on the anatomic location of the tunnel, a template is created for attachment to a surgical guide. The template includes at least one aperture for directing a drill inserted through the surgical guide to drill the tunnel at the anatomic location.

In further examples, determining the at least one anatomic insertion point of the ligament includes determining the at least one anatomic insertion point on a series of 2-dimensional images obtained from the electronic image data. In examples, determining the at least one anatomic insertion point of the ligament includes determining the at least one anatomic insertion point on the 3-D model using the at least one anatomic insertion point on the series of 2-dimensional images. In examples, the at least one bone is a femur or a tibia, and the ligament is an anterior cruciate ligament or at least one of an anteromedial or posterolateral bundle. In examples, creating the template comprises creating the template by additive manufacturing. In examples, a surface of the template comprises retention features for securing the template to the at least one bone. In examples, the template is comprised of plastic. In examples, the electronic image data is obtained using magnetic resonance imaging (MRI).

Examples of a template for attachment to a surgical guide of this disclosure include a template formed by the method of obtaining electronic image data of a joint, including at least one bone, of a patient; using the electronic image data, creating a 3-D model of the at least one bone; using the 3-D model, determining at least one anatomic insertion point of a ligament on the at least one bone; based on the at least one anatomic insertion point, determining an anatomic location of a tunnel through the at least one bone for housing a graft; and, based on the anatomic location of the tunnel, creating the template for attachment to the surgical guide.

Examples of a method for simulating reconstructive surgery of a ligament using electronic image data of this disclosure, the method at least partially executed by a processor within a computing system, include obtaining electronic image data of a joint, including at least one bone, of a patient; creating a 3-D model of the at least one bone using the electronic image data; determining at least one anatomic insertion point of a ligament on the at least one bone based on the 3-D model; determining an anatomic location of a tunnel through the at least one bone for housing a graft based on the at least one anatomic insertion point; and mapping and superimposing, using augmented reality, the anatomic location of the tunnel on a real-time image of the at least one bone on a display device.

In further examples, determining the at least one anatomic insertion point of the ligament comprises determining the at least one anatomic insertion point on a series of 2-dimensional images obtained from the electronic image data. In examples, determining the at least one anatomic insertion point of the ligament comprises determining the at least one anatomic insertion point on the 3-D model using the at least one anatomic insertion point on the series of 2-dimensional images. In examples, the at least one bone is a femur or a tibia and the ligament is an anterior cruciate ligament or at least one of an anteromedial or a posterolateral bundle. In examples, the electronic image data is obtained using magnetic resonance imaging (MRI). In examples, superimposing the anatomic location of the tunnel on the real-time image of the at least one bone comprises superimposing a silhouette of the at least one anatomic insertion point on a real-time image of a femoral condyle. In examples, the method further includes mapping the at least one anatomic insertion point onto the 3-D model of the at least one bone and displaying the 3-D model of the at least one bone on a portion of the display device.

These and other features and advantages is apparent from a reading of the following detailed description and a review of the associated drawings. It is to be understood that both the foregoing general description and the following detailed description are explanatory only and are not restrictive of aspects as claimed.

### BRIEF DESCRIPTION OF THE DRAWINGS

The disclosure is more fully understood by reference to the detailed description, in conjunction with the following figures, wherein:

FIG. 1 is a schematic illustration of a patient-specific surgical instrument of this disclosure;

FIG. 2 is an illustration of a knee joint of a patient in a two-dimensional view;

FIG. 3 is an illustration of a 3-D model of the knee joint of the patient;

FIGS. 4A-C are detailed illustrations of a patient-specific template for use with the surgical instrument of this disclosure;

FIGS. 5A-C illustrate a method of superimposing images onto a real-time display of a knee joint of a patient during a ligament reconstruction; and

FIGS. 6A and 6B illustrate another 3-D model of the femoral bone of a knee joint of the patient and its corresponding ligament insertion points.

### DETAILED DESCRIPTION

In the description that follows, like components have been given the same reference numerals, regardless of whether they are shown in different examples. To illustrate example(s) in a clear and concise manner, the drawings may not necessarily be to scale and certain features may be shown in somewhat schematic form. Features that are described and/or illustrated with respect to one example may be used in the same way or in a similar way in one or more other examples and/or in combination with or instead of the features of the other examples.

As used in the specification and claims, for the purposes of describing and defining the invention, the terms “about” and “substantially” are used to represent the inherent degree of uncertainty that may be attributed to any quantitative comparison, value, measurement, or other representation. The terms “about” and “substantially” are also used herein to represent the degree by which a quantitative representation may vary from a stated reference without resulting in a change in the basic function of the subject matter at issue. “Comprise,” “include,” and/or plural forms of each are open ended and include the listed parts and can include additional parts that are not listed. “And/or” is open-ended and includes one or more of the listed parts and combinations of the listed parts.

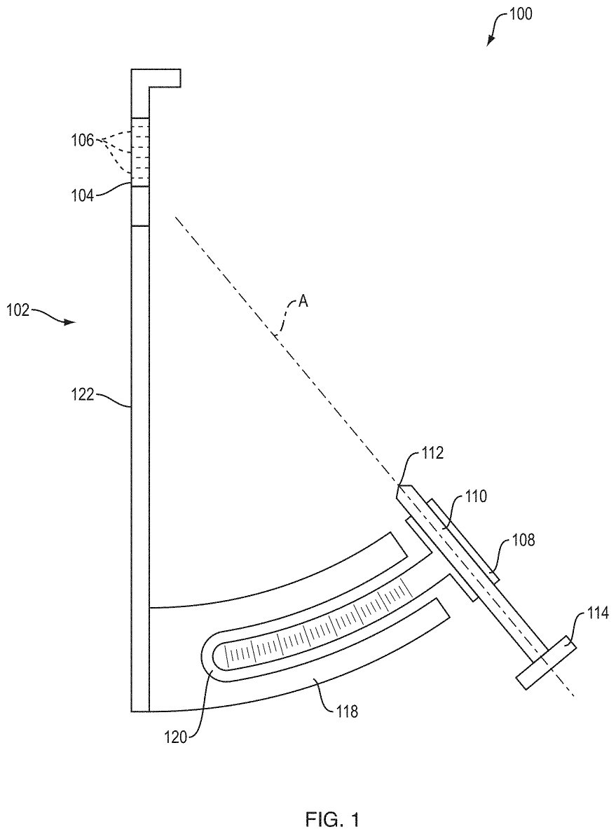

Referring now to FIGS. 1-3, a surgical instrument 100 for ligament repair of this disclosure, as well as methods of making the instrument 100 using patient-specific data, are illustrated. As shown in FIG. 1, the instrument 100 generally comprises a reusable guide 102 and an attachable template 104 with apertures 106 corresponding to the footprint of the ACL. The apertures 106 may also correspond to the footprints of the anteromedial (AM) bundle and the posterolateral (PL) bundle of the ACL, as further described below. Alternatively, it is contemplated by this disclosure that the instrument 100 could be designed for the insertion of the posterior cruciate ligament (PCL) or other ligaments.

Still referring to FIG. 1, examples of the guide 102 can include a housing 108 for retaining an insertion member 110. Examples of the insertion member 110, known in the art as a “bullet,” have an aimer tip 112 and an insertion knob 114 for directing a drill (not shown) inserted through the insertion member 110 along an insertion axis A to an anatomic insertion point at a surgical site, such as a bone tunnel location on a femur. In examples, the housing 108 couples to an aimer arm 118 via a slot 120 for sliding movement therein, and may have an arc shape for arcuate movement. A guide arm 122 for attachment to the template 104 of this disclosure couples to the aimer arm 118, and may have a hinged connection (not shown) for rotation of the guide arm 122 and the template 104 in the plane defined by the aimer arm 118 and the insertion member 110. The guide arm 122 may be straight, as shown, or may be curved toward the insertion axis A. The apertures 106 in the template 104 define the drilling footprint at the surgical site, thus providing an indication of the diameter and anatomic location of the resulting bone tunnel, while the insertion axis A indicates the path of the bone tunnel through the apertures 106 in the template 104. In examples, a length of the guide arm 122 could be adjustable to ensure that the insertion axis A passes through the desired apertures 106 on the template 104. Other non-limiting examples of the guide 102 are described in U.S. Pat. No. 9,078,675 to Smith & Nephew, Inc., incorporated herein by reference in its entirety.

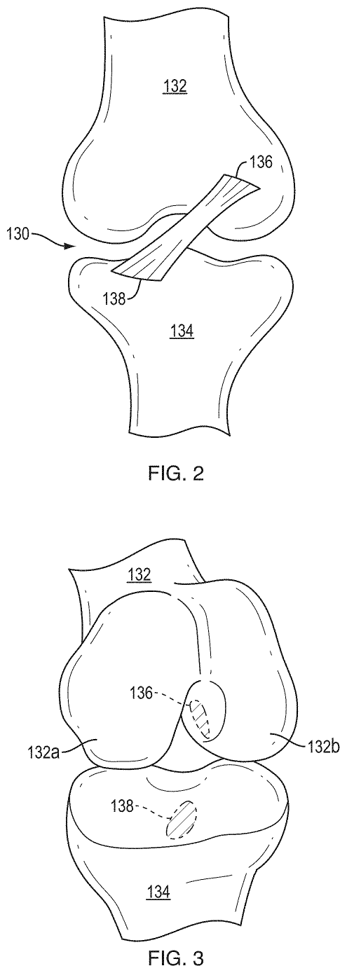

Turning now to FIG. 2, a knee joint 130 of a patient, including a femur 132 and a tibia 134, is illustrated in a two-dimensional view. Initially, to form the surgical instrument 100 of FIG. 1, a series of pre-operative, two-dimensional electronic images of the knee joint 130 are created using a series of electronic image data. The electronic image data may be derived from computed tomography (CT) or magnetic resonance imaging (MRI). However, soft tissues and their corresponding insertion points can generally be determined more accurately using MM. The series of 2-D images may include the two femoral condyles 132a,b (FIG. 3) about 10-15 cm from the knee joint line. Once the images of the knee joint 130 are generated, the femoral ACL footprint 136 and the tibial ACL footprint 138 are identified on the series of 2-D images. The anatomic footprints 136, 138 may be identified by their unique contour on the bone surface or by identifying ligament fiber remnants in their respective locations. Next, a 3-D model of the femur 132 and the tibia 134 is created from the series of 2-D images. As shown in FIG. 3, the 3-D anatomic locations of ACL footprints 136, 138 are then defined on the 3-D bone model from the identified ACL footprints 136, 138 on the 2-D images. The data obtained from the geometry of the ACL footprint (and potentially the geometry of the patient's lateral femoral condyle 132b) can then be used to create the patient-specific template 104 of this disclosure with specifications tailored to the patient's ligament footprints 136, 138 for use during the patient's ligament reconstruction procedure.

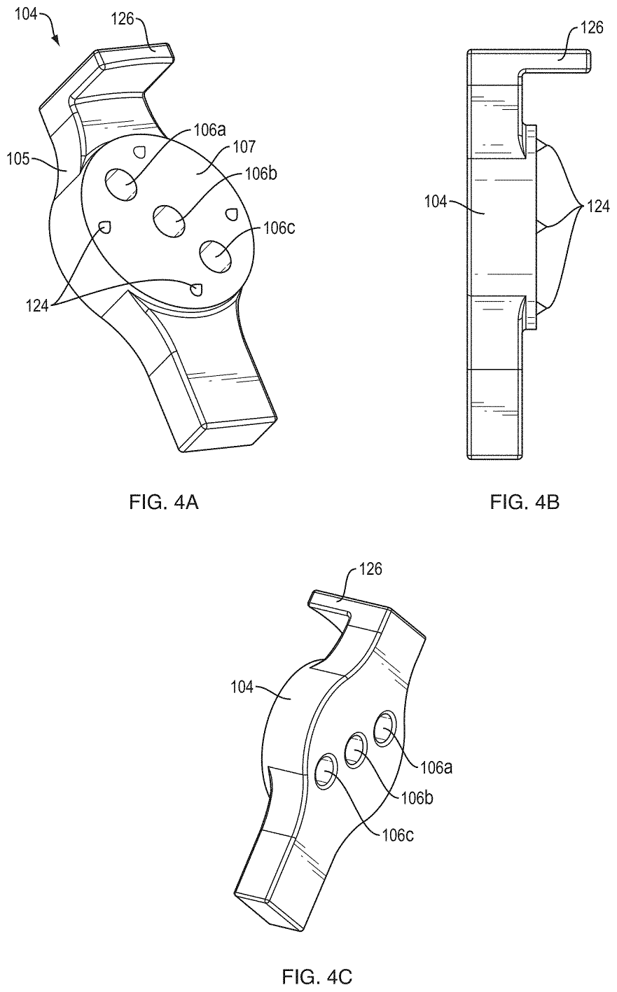

Turning now to FIGS. 4A-C, examples of the patient specific template 104 are shown in detailed views. The template 104 can be attached to a currently available guide 102, such as the guide 102 of FIG. 1, and used during the ligament reconstruction. For example, the template 104 could be configured for a snap-fit to the guide 102. In examples, the template 104 may be created using additive manufacturing (i.e., “3-D printing”) and is comprised of a plastic. Examples of the template 104 include an elongated, flattened body 105 including a wider, circular area 107 that is slightly raised toward the insertion axis A (FIG. 1). A surface of the circular area 107 facing the insertion axis A may include spikes 124 or other retention features for securing the template 104 to a surface of the bone. The circular area 107 also defines the apertures 106a,b,c, corresponding to the footprints of the ACL or its bundles. The body 105, including the circular area 107, provide a low profile to facilitate insertion between anatomical members. The body 105 of the template 104 may be planar, as shown, or may be curved toward the insertion axis A. A length of the template 104, as well as the size of and distance between the apertures 106a,b,c can vary based on the anatomy of the individual patient. An end of the body 105 opposite the guide arm 122 comprises an end hook 126 angled toward the insertion axis A. When the end hook 126 of the template 104 is placed behind the posterior wall of the patient's femoral notch and the guide 102 is held parallel to tibial plateau, aperture 106b of the template 104 indicates the anatomic insertion of the ACL. Similarly, the apertures 106a and 106c indicate the anatomic insertion of the AM/PL bundles, respectively. Once proper anatomic and/or functional positioning of the template 104 is achieved, the ligament tunnel can be created using the guide 102.

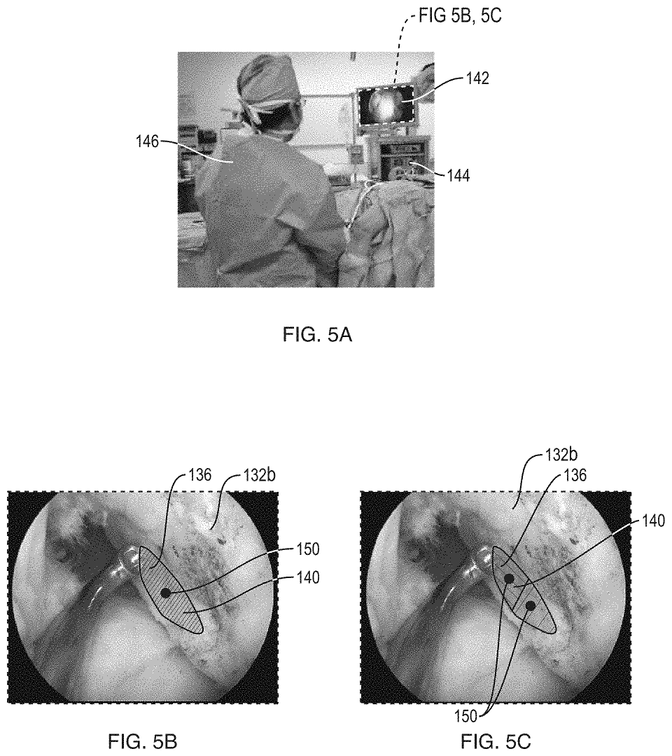

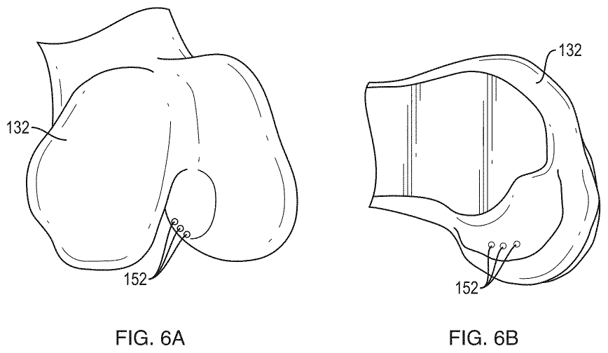

In alternative examples, shown in FIGS. 5A-C, the femoral ACL footprint 136 which was identified on the 3-D model of the patient's knee joint 130 is registered and mapped onto a real-time image of the patient's femoral condyle 132b during the ligament reconstruction. For example, using augmented reality (AR), a silhouette 140 of the ACL footprint 136 can be shown on the computer display 142 of the arthroscopy tower 144 overlapped on the real-time image of the femoral condyle 132b, such that the silhouette 140 does not block the view of the surgeon 146. The silhouette 140 can provide the surgeon 148 with a reference point to decide the final anatomic location of the ACL footprint 136 before placing the graft tunnel. The insertion areas of the ACL footprint 136 can be presented in two modes and surgeon can switch between these modes. For example, the modes may include a single bundle mode for the ACL (FIG. 5B) or a double bundle mode for the AM/PL bundles (FIG. 5C). The centroid 150 of each insertion area can be added to the view as a turn on/off option. Additionally, as shown in FIGS. 6A and 6B, the anatomic insertion points 152 can be mapped onto a 3-D model of the femur 132 of the individual patient using augmented reality for an overall visualization of the anatomic insertion points 152 and as an extra check point. The 3-D model can be displayed in a corner of the computer display 142.

One skilled in the art will realize the disclosure may be embodied in other specific forms without departing from the spirit or essential characteristics thereof. The foregoing examples are therefore to be considered in all respects illustrative rather than limiting of the disclosure described herein. Scope of the disclosure is thus indicated by the appended claims, rather than by the foregoing description, and all changes that come within the meaning and range of equivalency of the claims are therefore intended to be embraced therein.

1. A method of making a surgical instrument for ligament reconstruction/repair, the method comprising:
obtaining electronic image data of a joint, including at least one bone, of a patient;
using the electronic image data, creating a 3-D model of the at least one bone;
using the 3-D model, determining at least one anatomic insertion point of a ligament on the at least one bone;
based on the at least one anatomic insertion point, determining an anatomic location of a tunnel through the at least one bone for housing a graft; and
based on the anatomic location of the tunnel, creating a template for attachment to a surgical guide, the template including at least one aperture for directing a drill inserted through the surgical guide to drill the tunnel at the anatomic location.

2. The method of claim 1, wherein determining the at least one anatomic insertion point of the ligament comprises determining the at least one anatomic insertion point on a series of 2-dimensional images obtained from the electronic image data.

3. The method of claim 2, wherein determining the at least one anatomic insertion point of the ligament comprises determining the at least one anatomic insertion point on the 3-D model using the at least one anatomic insertion point on the series of 2-dimensional images.

4. The method of claim 1, wherein the at least one bone is a femur or a tibia.

5. The method of claim 1, wherein the ligament is an anterior cruciate ligament.

6. The method of claim 1, wherein the ligament is at least one of an anteromedial or posterolateral bundle.

7. The method of claim 1, wherein creating the template comprises creating the template by additive manufacturing.

8. The method of claim 1, wherein a surface of the template comprises retention features for securing the template to the at least one bone.

9. The method of claim 1, wherein the template is comprised of plastic.

10. The method of claim 1, wherein the electronic image data is obtained using magnetic resonance imaging (MRI).

11. A template for attachment to a surgical guide formed by the method of claim 1.

12. A method for simulating reconstructive surgery of a ligament using electronic image data, the method at least partially executed by a processor within a computing system, the method comprising:
obtaining electronic image data of a joint, including at least one bone, of a patient;
creating a 3-D model of the at least one bone using the electronic image data;
determining at least one anatomic insertion point of a ligament on the at least one bone based on the 3-D model;
determining an anatomic location of a tunnel through the at least one bone for housing a graft based on the at least one anatomic insertion point; and
mapping and superimposing, using augmented reality, the anatomic location of the tunnel on a real-time image of the at least one bone on a display device.

13. The method of claim 12, wherein determining the at least one anatomic insertion point of the ligament comprises determining the at least one anatomic insertion point on a series of 2-dimensional images obtained from the electronic image data.

14. The method of claim 12, wherein determining the at least one anatomic insertion point of the ligament comprises determining the at least one anatomic insertion point on the 3-D model using the at least one anatomic insertion point on the series of 2-dimensional images.

15. The method of claim 12, wherein the at least one bone is a femur or a tibia.

16. The method of claim 12, wherein the ligament is an anterior cruciate ligament.

17. The method of claim 12, wherein the ligament is at least one of an anteromedial or a posterolateral bundle.

18. The method of claim 12, wherein the electronic image data is obtained using magnetic resonance imaging (MRI).

19. The method of claim 12, wherein superimposing the anatomic location of the tunnel on the real-time image of the at least one bone comprises superimposing a silhouette of the at least one anatomic insertion point on a real-time image of a femoral condyle.

20. The method of claim 12, further comprising:
mapping the at least one anatomic insertion point onto the 3-D model of the at least one bone; and
displaying the 3-D model of the at least one bone on a portion of the display device.
